(19) United States
(12) Patent Application Publication
(10) Pub. No.: US 2004/0142048 A1
(43) Pub. Date: Jul. 22, 2004

(54) Compositions based on proanthocyanadin-catechin synergies for prevention and treatment of cancer

(76) Inventors: Dorothy M. Morre, West Lafayette, IN (US); D James Morre, West Lafayette, IN (US)

Correspondence Address: JONES DAY, 222 EAST 41ST ST, NEW YORK, NY, 10017, US

(21) Appl. No.: 10/468,911

(22) PCT Filed: Feb. 22, 2002
(86) PCT No.: PCT/US02/05294

(51) Int. Cl. A61K 35/78; A61K 31/7048; A61K 31/353

(52) U.S. Cl. 424/729; 514/27; 514/456

(57) ABSTRACT

The invention described herein encompasses methods and compositions of preventing or treating cancer comprising the administration of a combination of catechins and proanthocyanadins. Compositions of catechins include but not limited to, epigallocatechin gallate (EGCg), epicatechin (EC), epicatechin gallate (ECG), epigallocatechin (EGC). Compositions of proantho-cyanains include, but are not limited to, blue fruit extracts. The unique compositions of the invention contain various combinations of the catechins and proanthocyanadins, in combination with each other or other therapeutic agents and are used to treat primary and metastatic cancers in humans. The invention also encompasses various modes of administration of the therapeutic compounds, including formulations which may be used as a dietary or nutritional supplement or as a therapeutic compound.

This application claims benefit of U.S. provisional application serial No. 60/270,561, filed Feb. 22, 2001.

### 1. INTRODUCTION

The present invention relates to novel methods and compositions which utilize catechins, including but not limited to, epigallocatechin gallate (EGCg), epicatechin (EC), epicatechin gallate (ECG), and epigallocatechin (EGC), which are found in varying levels in tea leaves, in combination with proanthocyanadins, including but not limited to, blue fruit extracts. The compositions of the invention contain various amounts of the catechins and proanthocyanadins, and optionally, other therapeutic agents. The invention also encompasses the varying modes of administration of the catechins and proanthocyanadins as a dietary or nutritional supplement or as a therapeutic compound.

### 2. BACKGROUND OF THE INVENTION

### 2.1. Tea Catechins

Tea is generally in the form of black, oolong, and green tea, all originating from the tea plant, Camellia sinensis. Tea is cultivated in approximately thirty countries worldwide, and is consumed globally. Although the level of tea consumption varies around the world, it is believed that tea consumption is second only to water (Ahmad et al., 1998, Nutrition and Chemical Toxicity, John Wiley and Sons, Sussex, England, pp. 301-343). Black tea is consumed predominantly in Western and some Asian countries and green tea is consumed predominantly in China, Japan, India, and a few countries in North Africa and the Middle East (Ahmad et al., 1998, Nutrition and Chemical Toxicity, John Wiley and Sons, Sussex, England, pp.301-343).

Green tea has been prized as a traditional tonic and has been widely consumed in East Asia. Recent studies have attempted to link green tea to antioxidant benefits including protection against the damage caused by cigarette smoke, pollution, stress, and other toxins (for an overview, see e.g., Mitscher, 1998, The Green Tea Book, Avery Publishing Group, Garden City Park, N.Y. and Weisburger, 1997, Can. Lett. 114:315-317).

An empirical link between green tea and its cancer prevention properties was made in the late 1980s (Khan et al., 1988, Can. Lett. 42:7-12 and Wang et al., 1989, Carcinogenesis 10:411-415). Epidemiological studies show that cancer onset of patients in Japan who had consumed ten cups of green tea per day was 8.7 years later among females and 3 years later among males, compared with patients who had consumed under three cups per day (Fujiki et al., 1998, Mutation Res. 402:307-310). As such, a possible relationship between high consumption of green tea and low incidence of prostate and breast cancer in Asian countries where green tea consumption is high has been postulated (Liao et al., 1995, Can. Lett. 96:239-243 and Stoner and Mukhtar, 1995, J. Cell. Biochem. 22:169-180). However, because of the many variables in lifestyle inherent to such a study, a definitive link between green tea and its cancer prevention effects could not be concluded.

Scientists have now identified many of the natural substances in green tea that may provide the majority of its health benefits. One class of chemicals that has attracted much study is the polyphenols, also known as catechins.

The polyphenols describe a class of substituted phenolic compounds that are known as flavanols or catechins. The polyphenols in green tea that have been identified are catechin (C), epicatechin (EC), gallocatechin (GC), gallocatechin gallate (GCG), epigallocatechin (EGC), epicatechin gallate (ECG), and epigallocatechin gallate (EGCg) (FIG. 1). In addition, caffeine, theobromine, theophylline, and phenolic acids, such as gallic acid, are also present as constituents of green tea in smaller quantities than the polyphenols (Ahmad et al., 1998, Nutrition and Chemical Toxicity, John Wiley and Sons, Sussex, England, pp. 301-343).

Epigallocatechin gallate (EGCg), the major catechin in green tea, has been the focus of many studies to determine if it is responsible for the antioxidant and anti-carcinogenic effects of green tea, as reviewed by Ahmad and Mukhtar, 1999, Nutr. Rev. 57:78-83. The administration of a pharmacologically effective amount of EGCg has been alleged to reduce the incidence of lung cancer in a mammal (U.S. Pat. No. 5,391,568). A bioavailability study showed that frequent green tea consumption results in high levels of EGCg in various body organs, suggesting that green tea consumption may protect against cancers localized to different sites of the body (Sugunama et al., 1998, Carcinogenesis 19:1771-1776).

EGCg has been implicated in blocking DNA transcription of a number of genes in cancer cell lines. For example, in the human epidermal carcinoma cell line A431, EGCg inhibits the DNA and protein synthesis of the growth factor receptors epidermal growth factor receptor (EGF-R), platelet-derived growth factor receptor (PDGF-R), and fibroblast growth factor receptor (FGF-R) (Liang et al., 1997, J. Cell. Biochem. 67:55-65). EGCg has also been implicated in blocking transcription of nitric oxide (NO) synthase by inhibiting the binding of transcription factor NFκB to the NO synthase promotor (Lin and Lin, 1997, Mol. Pharmacol. 52:465-472 and Chan et al., 1997, Biochem. Pharmacol. 54:1281-1286). In the tumor cell line JB6, EGCg inhibits AP-1 transcriptional activity (Dong et al., 1997, Can. Res. 57:4414-4419). These results suggest that EGCg may prevent cancer at the level of gene transcription, i.e., by blocking the DNA synthesis of genes involved in signal transduction pathways.

Further, the focus of many other studies has been the effect of EGCg on apoptosis, or programmed cell death. Apoptosis differs from necrosis, and is regarded as an ideal mechanism for the elimination of cells. Studies have shown that several anti-cancer preventative agents may induce apoptosis, and conversely, several tumor-promoting agents inhibit apoptosis (Wright et al., 1994, FASEB J 8:654-660 and Ahmad and Mukhtar, 1999, Nutr. Rev. 57:78-83).

Much of the prior work in the art has attempted to determine what, if any, effect EGCg has on the growth inhibition and apoptosis induction of cancer cells. A differential growth inhibitory effect was reported in human colorectal cancer cells CaCo-2, breast cancer cells Hs578T, and their non-cancer cell counterparts (Ahmad and Mukhtar, 1999, Nutr. Rev. 57:78-83). EGCg has been implicated in the growth arrest and subsequent induction of apoptosis following cell growth inhibition has been shown in virally transformed fibroblast cells WI138, human epidermal carcinoma cells A431, lung cancer tumor cells H611, prostate cancer cell lines LNCaP, PC-3, and DU145, human carcinoma keratinocytes HaCaT, and mouse lymphoma cells LY-R (Chen et al., 1998, Can. Lett. 129:173-179; Ahmad et al., 1997, J. of the Nat. Can. Inst. 89:1881-1886; Yang et al., 1998, Carcinogenesis 19:611-616; Paschka et al., 1998, Can. Lett. 130:1-7; and Ahmad and Mukhtar, 1999, Nutr. Rev. 57:78-83). In studies where the apoptotic response was studied in cancer cells versus their non-cancer counterparts, e.g., human carcinoma keratinocytes HaCaT versus normal human epidermal keratinocytes, the apoptotic response to EGCg was reported to be specific to the cancer cells (Ahmad et al., 1997, J. Nat. Can. Inst. 89:1881-1886).

It has been suggested that EGCg induced apoptosis may result from either cell cycle arrest and/or H₂O₂ production (Ahmad et al., 1997, J. Nat. Can. Inst. 89:1881-1886; Fujiki et al., 1998, Mutat. Res. 402:307-310; and Yang et al., 1998, Carcinogenesis 19:611-616). EGCg may be involved in the growth regulation of human epidermal carcinoma cells A431 by causing cell cycle arrest of the G₀ to G₁ phase (Ahmad et al., 1997, J. Nat. Can. Inst. 89:1881-1886). EGCg has also been implicated in phase arrest between G₂ to M phase of the cell cycle in human lung cancer cells (Fujiki et al., 1998, Mutat. Res. 402:307-310). In the EGCg induced inhibition of human lung cancer cells, it was suggested that the tumor necrosis factor (TNF) α pathway that is the mode of action of EGCg. Alternatively, the EGCg-induced apoptosis of the lung cancer tumor cells H611 is inhibited by catalase, suggesting the H₂O₂ production as a probable cause of apoptosis (Yang et al., 1998, Carcinogenesis 19:611-616).

Despite the above studies, the efficacy of EGCg as a single agent therapy for the prevention of cancer is still unclear. Moreover, the efficacy of EGCg as a therapeutic drug to treat or reverse cancer in a patient is unknown.

Although the focus of much of the prior research has been on EGCg, the putative biological functions of some of the other catechins has been examined. For example, both epicatechin gallate (ECG) and epigallocatechin (EGC) have been reported to be as effective as EGCg in inducing apoptosis of human epidermal carcinoma cells A431 at similar concentrations, whereas epicatechin (EC) did not show a similar effect (Ahmad et al., 1997, J. of the Nat. Can. Inst. 89:1881-1886). Growth inhibition in lung tumor cell lines H661 and H1299 was also observed with EGCg and EGC, whereas ECG and EC were less effective (Yang et al., 1998, Carcinogenesis 19:611-616).

Catechins have been implicated in growth inhibition of the human lung cancer cell line PC-9, with the order of catechin potency being reported as EGCg=ECG>EGC [symbol] EC (Okabe et al., 1993, Jpn. J. Clin. Oncol. 23:186-190). It has also been demonstrated that catechin combinations of EGCg and EC, ECG and EC, and EGC and EC induce apoptosis of the human lung cancer cell line PC-9 in vitro (Suganuma et al., 1999, Can. Res. 59:44-47). EC is thought to enhance incorporation of EGCg into the cells, which is thought to inhibit TNF α release resulting in the induction of apoptosis (Suganuma et al., 1999, Can. Res. 59:44-47).

Green tea extract, an important source of EGCg, has previously been reported to enhance the effect of the anti-cancer agents, e.g., adriamycin and doxorubicin (Sugiyama and Sadzuka, 1998, Can. Lett. 133:19-26 and Sadzuka et al., 1998, Clin. Can. Res. 4:153-156). Green tea in combination with adriamycin inhibits tumor growth in M5076 ovarian sarcoma cells, whereas adriamycin alone does not inhibit tumor growth in M5076 ovarian sarcoma cells (Sugiyama and Sadzuka, 1998, Can. Lett. 133:19-26). A similar effect is observed with green tea extract and doxorubicin on the same M5076 ovarian sarcoma cell line. Green tea extract, in combination with doxorubicin, also enhances the inhibitory growth effect on Ehrlich ascites carcinoma tumors in tumor-bearing mice, presumably by increasing the concentration of doxorubicin concentration in the tumor, but not in normal tissue (Sadzuka et al., 1998, Clin. Can. Res. 4:153-156).

EGCg has also been shown to enhance the effect of cancer prevention drugs in vitro. For example, EGCg has been shown to enhance the apoptotic effect of sulindac and tamoxifin, presumably by EGCg enhancing the intracellular concentration of the cancer prevention drugs. (Suganuma et al., 1999, Can. Res. 59:44-47). Both sulindac and tamoxifin induce apoptosis of human cancer cells and inhibit TNF α release from BALB/c-3T3 cells (Piazza et al., 1995, Can. Res. 55:3110-3116; Chen et al., 1996, J. Cell. Biochem. 61:9-17; and Sugunama et al., 1996, Can. Res. 56:3711-3715).

### 2.2. Proanthocyanadins

Proanthocyanadins are dietary sources of polyphenols. As described by Fine in a review article, which is incorporated by reference in its entirety, considerable recent research has explored therapeutic applications of oligomeric proanthocyanidin complexes, which are naturally occurring plant metabolites widely available in fruits, vegetables, nuts, seeds, flowers, and bark and are primarily known for their antioxidant activity (Fine, 2000, Altern Med Rev 5(2):144-51).

Oligomeric proanthocyanidins are naturally occurring antioxidants widely available in fruits, vegetables, nuts, seeds, flowers and bark, which have been reported to possess a broad spectrum of biological, pharmacological and therapeutic activities against free radicals and oxidative stress. As described in Bagchi et al., a novel grape seed proanthocyanidin extract has been shown to provide excellent protection against oxidative stress and free radical-mediated tissue injury (Bagchi et al., 2000, Toxicology 148(2-3):187-97). Bomser et al. has disclosed that a polyphenolic fraction from grape seeds results in the inhibition of TPA-induced tumor promotion in CD-1 mouse epidermis (Bomser et al., 1999, Cancer Lett. 132(2):151-7).

Proanthocyanadins are found in the extracts of blue fruits such as, but not limited to, wild blackberries (Rubus spp.) (Rosaceae), blue berries (Vaccinium sp.) (Ericaceae), elderberry (Sambucus canadensis) (Caprifoliaceae), choke cherry (Prunus virginiana) (Rosaceae), pokeberry (Phytolacca americana) (Phytolaccaceae), wild grape (fox grape) (Vitis labrusca) (Vitaceae), and green briar (Smilax glauca) (Liliaceae). As described herein, proanthocyanadins, in combination with the green tea polyphenols, was shown to have potential utility for the prevention and treatment of cancer.

### 2.3. NADH Oxidase

A unique plasma membrane NADH oxidase (NOX), a unique cell surface protein with hydroquinone (NADH) oxidase and protein disulfide-thiol interchange activities that is responsive to hormone and growth factors has been identified (Brightman et al., 1992, Biochim. Biophys. Acta 1105:109-117; Morré, 1994, J. Bioenerg. Biomemb. 26:421-433; and Morré, 1998, Plasma Membrane Redox Systems and their Role in Biological Stress and Disease, Klewer Academic Publishers, Dordrecht, The Netherlands, pp. 121-156). Further, a hormone-insensitive and drug-responsive form of NOX designated tNOX which is specific to cancer cells has been reported (Bruno et al., 1992, Biochem. J. 284:625-628; Morré and Morré, 1995, Protoplasma 184:188-195; Morré et al., 1995, Proc. Natl. Acad. Sci. U.S.A. 92;1831-1835; Morré et al., 1995, Biochirn. Biophys. Acta 1240:11-17; Morré et al., 1996, Eur. J. Can. 32A: 1995-2003; and Morré et al., 1997, J. Biomemb. Bioenerg. 29:269-280).

Because the NOX protein is located at the external plasma membrane surface and is not transmembrane, a functional role as an NADH oxidase is not considered likely (Morré, 1994, J. Bioenerg. Biomemb. 26:421-433; DeHahn et al., 1997, Biochirn. Biophys. Acta 1328:99-108; and Morré, 1998, Plasma Membrane Redox Systems and Their Role in Biological Stress and Disease, Klewer Academic Publishers, Dordrecht, The Netherlands, pp. 121-156). While the oxidation of NADH provides a basis for a convenient method to assay the activity, the ultimate electron physiological donor is most probably hydroquinones with specific activities for hydroquinone oxidation greater than or equal to that of NADH oxidation and/or protein thiol-disulfide interchange (Kishi et al., 1999, Biochim. Biophys. Acta 1412:66-77).

CNOX was originally defined as a drug-indifferent constitutive NADH oxidase activity associated with the plasma membrane of non-transformed cells that was the normal counterpart to tNOX (Morré, 1998, Plasma Membrane Redox Systems and Their Role in Biological Stress and Disease, Kiewer Academic Publishers, Dordrecht, The Netherlands, pp. 121-156). Indeed, a 36 kD protein isolated from rat liver and from plants has NOX activity that is unresponsive to tNOX inhibitors (Brightman et al., 1992, Biochim. Biophys. Acta 1105: 109-117).

While cancer cells exhibit both drug-responsive and hormone and growth factor-indifferent (tNOX) as well as drug inhibited and hormone and growth factor dependent (CNOX) activities, non-transformed cells exhibit only the drug indifferent hormone- and drug-responsive CNOX. Among the first descriptions of so-called constitutive or CNOX activity of non-transformed cells and tissues was where the activity of rat liver plasma membranes was stimulated by the growth factor, diferric transferrin (Sun et al., 1987, J. Biol. Chem. 262:15915-15921). Subsequent work demonstrated that the observed NADH oxidation was catalyzed by a unique enzyme exhibiting responsiveness to several hormones and growth factors (Bruno et al., 1992, Biochem J. 284:625-628). Unlike mitochondrial oxidases, the hormone-stimulated NADH oxidase activity of rat liver plasma membranes is not inhibited by cyanide (Morré, 1994, J. Bioenerg. Biomemb. 26: 421433). The enzyme also was distinguished from other oxidase activities by its response to several common oxidoreductase inhibitors, e.g., catalase, azide and chloroquine, as well as to various detergents e.g., sodium cholate, Triton X-100 and CHAPS (Morré and Brightman, 1991, J. Bioenerg. Biomemb. 23:469-489 and Morré et al., 1997, J. Biomemb. Bioenerg. 29:269-280). Like tNOX of cancer cells, CNOX is a unique membrane-associated protein that is capable of oxidizing NADH but has an activity which is modulated by hormones and growth factors.

### 2.4. Pathobiology of Cancer

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia.

Pre-malignant abnormal cell growth is exemplified by hyperplasia, metaplasia, or most particularly, dysplasia (for review of such abnormal growth conditions, see Robbins and Angell, 1976, Basic Pathology, 2d Ed., W.B. Saunders Co., Philadelphia, pp. 68-79) Hyperplasia is a form of controlled cell proliferation involving an increase in cell number in a tissue or organ, but without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

The neoplastic lesion may evolve clonally and develop an increasing capacity for invasion, growth, metastasis, and heterogeneity, especially under conditions in which the neoplastic cells escape the host's immune surveillance (Roitt, Brostoff, and Kale, 1993, Immunology, 3rd ed., Mosby, St. Louis, pp. 17.1-17.12).

There remains a need for treatment of cancer that does not have the adverse effects generally caused by non-selectivity, of conventional chemotherapeutic agents. None of the above studies, which are not to be construed as an admission that any of the above studies is prior art, have suggested the present mechanism by which the catechins are able to differentiate between cancer and non-cancer cells. Moreover, none of the studies evaluated the efficacy of catechins in combination with proanthocyanadins for the prevention or treatment of cancer. In contrast, the Inventors have identified a cancer-specific isoform of a unique plasma membrane NADH oxidase (tNOX) which is inhibited by the catechins and the proanthocyanadins. Furthermore, the studies cited supra have hypothesized that EGCg mediates its effects intracellularly, since EGCg incorporation into the cell seems to be a prerequisite for the inhibition of TNF α release. Inhibition of tNOX, an extracellular membrane-associated protein) by EGCg, and synergistically with other catechins, proanthocyanadins, and anti-cancer agents, results in the selective inhibition of cancer cell growth and ultimately, apoptosis of the cancer cells.

### 3. SUMMARY OF THE INVENTION

The invention encompasses formulations comprising catechins and proanthocyanadins in ratios that result in synergistic properties. The formulations are used as compositions for the prevention and treatment of cancer or as a dietary or nutritional supplement that protects white blood cells and maintains healthy blood levels. Specific therapeutic regimens, pharmaceutical compositions, and kits are also provided by the invention.

In one embodiment, the invention described herein comprises the administration of catechins in combination with proanthocyanadins, to a mammal as a dietary supplement. In a preferred embodiment, the mammal is a human.

In another embodiment, the invention described herein comprises the administration of a therapeutically effective amount of catechins in combination with proanthocyanadins, to a mammal in need of such therapy. In a preferred embodiment, the mammal is a human. In another embodiment, the invention further encompasses the use of additional therapeutic agent(s) in combination therapy to treat cancer.

In a specific embodiment, the catechins comprise epigallocatechin gallate (EGCg), epicatechin gallate (ECG), epigallocatechin (EGC), epicatechin (EC) or a combination thereof, in combination with one or more proanthocyanadins, such as, but not limited to, blue fruit extracts.

The disclosure is based, in part, on the discovery that catechins, proanthocyanadins, and other anti-cancer therapeutic agents, inhibit the activity of a cancer-specific protein, an isoform of NADH oxidase specific to cancer cells (tNOX). The inhibition of tNOX results in the inhibition of cell growth, and ultimately, apoptosis of the cancer cell, whereas normal cells (which lack tNOX but instead express the isoform CNOX) are less affected. Thus, the invention provides a potent therapeutic effect with reduced or no adverse effects on normal, healthy cells.

Significantly the effect of the catechins such as EGCg is reversible, i.e., if the EGCg is removed, cancer cells resume normal rates of growth. Other discoveries include: (1) EGCg is rapidly cleared from the blood and metabolized, (2) cancer cells must be inhibited from growing for 48 to 72 hours before EGCg-induced apoptosis occurs, and (3) when cancer cells are challenged with 10⁻⁷ M EGCg every two hours during the day, their growth is inhibited, but during the night normal cell growth resumes in the absence of further EGCg addition. Thus, one embodiment of the invention is directed to the administration of sustained release formulations so that a constant level of the catechins is maintained.

Particular compositions of the invention and their uses are described in the sections and subsections which follow.

### 3.1. Abbreviations

The catechins and target proteins defined herein are abbreviated as follows:

(±)-catechin C

(−)-epicatechin EC

gallocatechin GC

gallocatechin gallate GCG

(−)-epigallocatechin EGC

(−)-epicatechin gallate ECG

(−)-epigallocatechin gallate EGCg

nicotinamide adenine dinucleotide NADH

cell surface hydroquinone (NADH) oxidase with protein disulfide—thiol isomerase activity NOX

NOX present in both non-cancer and cancer cells CNOX

NOX specific to cancer cells tNOX

### 4. BRIEF DESCRIPTION OF THE DRAWINGS

FIGS. 1A-1B: Dose response of HeLa cell NADH oxidase activity (spectrophotometric assay) to juice of (A) greenbriar (Smilax glauca) and (B) pokeberry (Phytolacca americana) as being typical of the dose response relationships obtained.

FIGS. 2A-2B: Dose response of the HeLa cell NADH oxidase activity (spectrophotometric assay) to juice of (A) wild and (B) cultivated blackberry (Rubus spp.).

FIGS. 3A-3D: Dose response of the HeLa cell growth (96-well place assay) of juice of (A) pokeberry (Phytolacca americana), (B) green briar (Smilax glauca), (C) cultivated and (D) wild blackberry (Rubus spp.).

FIG. 4: Correlation between E dil₅₀ of tNOX inhibition with E dil₅₀ of growth inhibition (72 hours) of HeLa (human cervical carcinoma) cells in culture (96-well plate assay) for juices from eight blue fruits.

FIG. 5: Synergy (∘) between Tegreen™ (▴) and juice from wild blackberry (Δ) on growth inhibition of HeLa cells in culture determined by direct counting.

FIGS. 6A-6D: Combinations between Tegreen Tm and juices from different fruits on growth inhibition of HeLa cells in culture determined by direct counts. (A) choke cherry, (B) greenbriar, (C) pokeberry, and (D) cultivated blackberry.

### 5. DETAILED DESCRIPTION OF THE INVENTION

The present invention relates to compositions and methods for treatment and prevention of cancer.

The invention is based, in part, on the discovery that proanthocyanadins in blue fruits and catechins in tea, inhibit the activity of an isoform of NADH oxidase (tNOX) that is specific to cancer cells. The inhibition of tNOX results in the inhibition of cell growth, and ultimately, apoptosis of the cancer cell, whereas normal cells, which lack tNOX but express another isoform termed CNOX, are less affected. Accordingly, the invention provides a selective and potent therapeutic effect with reduced or no adverse effects on normal, healthy cells.

In one embodiment, the invention provides a composition comprising tea catechins and proanthocyanadins. In another embodiment, the invention provides a pharmaceutical composition comprising tea catechins, proanthocyanadins and a pharmaceutical carrier. In yet another embodiment, the invention provides a dietary supplement or nutritional composition comprising tea catechins and proanthocyanadins.

The invention further provides methods for preventing or treating cancer comprising administering compositions comprising tea catechins and proanthocyanadins. In yet another embodiment, the invention encompasses methods for preventing or treating cancer that comprises administering to a subject tea catechins adjunctively with proanthocyanadins such that both catechins and proanthocyanadins are present in vivo and in contact with cancer cells. In various embodiments, the cancer that is prevented or treated by the compositions and methods of the invention is a cancer that comprises cells that express tNOX.

The invention also provides methods for inhibiting the growth and/or proliferation of cancer cells and neoplastic cells comprising contacting the cancer cells and neoplastic cells with a composition comprising both tea catechins and proanthocyanadins. In yet another embodiment, the invention also encompasses methods for inhibiting the growth and/or proliferation of cancer and neoplastic cells comprising contacting the cells with tea catechins and with proanthocyanadins such that when the proanthocyanadins are contacted with the cells, the tea catechins are still providing the anti-cancer activity to the cells, and vice versa. In a preferred embodiment, the cancer cells and neoplastic cells that are inhibited by the catechin-proanthocyanadin mixtures comprise cells that express tNOX.

Particular compositions of the invention and their uses are described in the sections and subsections which follow.

### 5.1. Catechin-Proanthocyanadin Formulations

### 5.1.1. Catechin Formulations

In various embodiments of the invention, tea catechins are used either in the preparation of a composition of the invention that comprises both tea catechins and proantocyanidins, or in therapeutic or prophylactic methods in which tea catechins are administered adjunctively with proanthocyanidins. Various formulations of tea catechins can be used as above-described. The formulations used in the invention are based on green tea polyphenols typically found in green tea extracts which comprises 10-15% EGCg, 2-3% ECG, 2% EC, and 2-3% EGC (Suganuma et al., 1999, Can. Res. 59:44-47).

In one embodiment, the present invention provides for a formulation in which EGCg constitutes at least 30% of the total catechins. In a preferred embodiment, EGCg constitutes about 35% to about 45% of the total catechins. In a more preferred embodiment, EGCg constitutes about 40% of the total catechins.

Although the invention encompasses the use of a composition containing certain levels of EGCg alone, it is preferred that EGCg be used in combination with other catechins, more specifically, those described infra.

In another embodiment, the invention provides a formulation in which EGCg constitutes at least 30% of the total catechins and ECG constitutes at least 5% of the total catechins. In a preferred embodiment, EGCg constitutes about 35% to about 45% of the total catechins and ECG constitutes about 10% to about 20% of the total catechins. In a more preferred embodiment, EGCg constitutes about 40% of the total catechins and ECG constitutes about 15% of the total catechins.

In yet another embodiment, the invention provides a formulation in which EGCg constitutes at least 30% of the total catechins and EC constitutes at least 3% of the total catechins. In a preferred embodiment, EGCg constitutes about 35% to about 45% of the total catechins and EC constitutes about 3% to about 15% of the total catechins. In a more preferred embodiment, EGCg constitutes about 40% of the total catechins and EC constitutes about 7% of the total catechins.

In an additional embodiment, the invention provides a formulation in which EGCg constitutes at least 0.01% of the total catechins and EC constitutes an amount which is at least 10 fold greater than the EGCg content of the total catechins. The total catechins may or may not include additional catechins such as ECG, EGC, and C, described above. In a preferred embodiment, EC is present in an amount which is at least 100 fold greater than the EGCg content. In another preferred embodiment, the EC content is at least 1000 fold greater than the EGCg content.

In another embodiment, the amount of EGCg present in the catechin formulation is negligible.

In an additional embodiment, the invention provides a formulation in which EGCg constitutes at least 30% of the total catechins and EGC constitutes at least 1% of the total catechins. In a preferred embodiment, EGCg constitutes about 35% to about 45% of the total catechins and EGC constitutes about 2% to about 5% of the total catechins. In a more preferred embodiment, EGCg constitutes about 40% of the total catechins and EGC constitutes about 3% of the total catechins.

In an additional embodiment, the invention provides a formulation in which EGCg constitutes at least 30% of the total catechins, EC constitutes at least 3% of the total catechins, and ECG constitutes at least 5% of the total catechins. In a preferred embodiment, EGCg constitutes about 35% to about 45% of the total catechins, EC constitutes about 3% to about 15% of the total catechins, and ECG constitutes about 10% to about 20% of the total catechins. In a more preferred embodiment, EGCg constitutes about 40% of the total catechins, EC constitutes about 7% of the total catechins and ECG constitutes about 15% of the total catechins.

In yet another embodiment, the invention provides a formulation in which EGCg constitutes at least 30% of the total catechins, EC constitutes at least 3% of the total catechins, and EGC constitutes at least 1% of the total catechins. In a preferred embodiment, EGCg constitutes about 35% to about 45% of the total catechins, EC constitutes about 3% to about 15% of the total catechins, and EGC constitutes about 2% to about 5% of the total catechins. In a more preferred embodiment, EGCg constitutes about 40% of the total catechins, EC constitutes about 7% of the total catechins, and EGC constitutes about 3% of the total catechins.

In yet another embodiment, the invention provides a formulation in which EGCg constitutes at least 30% of the total catechins, EC constitutes at least 3% of the total catechins, ECG constitutes at least 5% of the total catechins, and EGC constitutes at least 1% of the total catechins. In a preferred embodiment, EGCg constitutes about 35% to about 45% of the total catechins, EC constitutes about 5% to about 15% of the total catechins, ECG constitutes about 10% to about 20% of the total catechins, and EGC constitutes 2% to about 5% of the total catechins. In a more preferred embodiment, EGCg constitutes about 40% of the total catechins, EC constitutes about 7% of the total catechins. ECG constitutes about 15% of the total catechins, and EGC constitutes about 3% of the total catechins.

In yet another embodiment, the invention provides a formulation in which EGCg constitutes at least 30% of the total catechins, EC constitutes at least 3% of the total catechins, ECG constitutes at least 5% of the total catechins, EGC constitutes at least 1% of the total catechins, and C constitutes at least 5% of the total catechins. In a preferred embodiment, EGCg constitutes about 35% to about 45% of the total catechins, EC constitutes about 5% to about 15% of the total catechins, ECG constitutes about 10% to about 20% of the total catechins, EGC constitutes 2% to about 5% of the total catechins, and C constitutes about 10% to about 20% of the total catechins. In a more preferred embodiment, EGCg constitutes about 40% of the total catechins, EC constitutes about 7% of the total catechins. ECG constitutes about 15% of the total catechins, EGC constitutes about 3% of the total catechins, and C constitutes about 15% of the total catechins.

In one embodiment, the invention provides a formulation which contains about 0.01% of EGCg of the total catechins. In another embodiment, the formulations contain about 0.1% of EGCg of the total catechins. In yet another embodiment, the formulation contains about 1.0% of EGCg of the total catechins. In yet another embodiment, the formulation contain about 5.0% of EGCg of the total catechins. In another embodiment, the formulation contains less than 10% of EGCg of the total catechins.

In another embodiment, the ratio of EC to EGCg concentration is about 10:1. In another embodiment, the ratio of EC to EGCg concentration is about 100:1. In yet another embodiment, the ratio of EC to EGCg concentration is about 1000:1.

In various embodiment, the level of caffeine in the formulation is generally less than about 5% and is preferably less than 0.5% of the polyphenols.

The catechin formulations described above can be made by infusing natural tea (see, e.g., Wang et al., 1994, Cancer Research 54:3428-3435 or U.S. Pat. No. 6,096,359, which is hereby incorporated by reference in its entirety) or by using tea concentrates that are commerically available (e.g., Tegreen™, Pharmanex, Brisbane, Calif.). The concentrations of the individual catechins in a formulation can be manipulated by adding purified catechins, which may be purchased (e.g., from Sigma, St. Louis, Mo.), or alternatively, purified from green tea by methods known to one of skill in the art (e.g., by high pressure liquid chromatography, “HPLC”). Many other methods for making tea-based compositions with altered levels of tea catechins can be used. For example, methods with generating tea formulations with reduced amounts of EGCg are disclosed in U.S. patent application Ser. No. 09/640,768, which is hereby incorporated by reference in its entirety.

### 5.1.2. Proanthocyanadin Formulations

In the present invention, proanthocyanadins are used either in the preparation of a composition of the invention that comprises both tea catechins and proantocyanidins, or in therapeutic or prophylactic methods in which proanthocyanidins are administered adjunctively with tea catechins. Various formulations of proanthocyanidin can be used as above-described.

Proanthocyanadins are dietary sources of polyphenols. Oligomeric proanthocyanidins are naturally occurring antioxidants widely available in fruits, vegetables, nuts, seeds, flowers and bark, which have been reported to possess a broad spectrum of biological, pharmacological and therapeutic activities against free radicals and oxidative stress. As described in U.S. Pat. Nos. 5,646,178 and 5,650,432, which are incorporated by reference in their entireties, proanthocyanadins are known to have a variable number of flavonoid units. The proanthocyanidins can have 2 to 28 flavonoid units, preferably 2 to 15 flavonoid units, and most preferably 2 to 11 flavonoid units. The flavonoid units include but are not limited to catechins, epicatechins, gallocatechins, galloepicatechins, flavanols, flavonols, flavandiols, leucocyanidins, leucodelphinidin anthocyanidins, or combinations thereof. The flavonoid units can be singly or double linked to each other. In various preferred embodiments, the proanthocyanadins used are present in blue fruits and include derivatives of proanthocyanadins, and related compounds resulting from the chemical breakdown of proanthocyanadins in blue fruits. As used herein, the term blue fruit extract refers to a preparation of blue fruits that comprises proanthocyanidins which can be obtained by methods such as, but not limited to, crushing, pressing, emulsifying, or chemically extracting a blue fruit.

According to the invention, the proanthocyanadin is derived from cultured blackberries (Rubus spp.) (Rosaceae). In a preferred embodiment, the proanthocyanadin is derived from wild blackberries (Rubus spp.) (Rosaceae). In another embodiment, the proanthocyanadin is derived from blue berries (Vaccinium sp.) (Ericaceae). In another embodiment, the proanthocyanadin is derived from elderberry (Sambucus canadensis) (Caprifoliaceae). In another embodiment, the proanthocyanadin is derived from choke cherry (Prunus virginiana) (Rosaceae). In another embodiment, the proanthocyanadin is derived from pokeberry (Phytolacca americana) (Phytolaccaceae). In another embodiment, the proanthocyanadin is derived from wild grape (fox grape) (Vitis labrusca) (Vitaceae). In another embodiment, the proanthocyanadin is derived from green briar (Smilax glauca) (Liliaceae). The proanthocyanadin can be obtained from a single or a mixture of blue fruits or blue fruit extracts. In yet another embodiment, the proanthocyanadin is derived from a concentrate of blue fruit, i.e., the concentrate contains more proanthocyanadins per unit mass than the fruit.

In another embodiment, as described in U.S. Pat. No. 5,646,178, which is incorporated by reference in its entirety, proanthocyanadins can be concentrated from blue fruits by first extracting active compounds from blue fruits with water or solvents such as, but not limited to, alcohols,acetone, acetonitrile, or ethyl acetate, or miscible mixtures of these solvents, water or methanol/water or acetone/water mixtures preferred, leaving a pulp or residue significantly depleted for the active compounds. Alternatively, the blue fruits may be extracted with relatively non-polar organic solvents such as but not limited to, hexane, heptane, cyclohexane, methylene chloride, chloroform, or large molecular weight alcohols that contain more than 8 carbon atoms, and the like. Such treatment extracts non-active materials, leaving a pulp or residue with increased concentrations of active substances. This pulp or residue may be further processed by extraction with more polar solvents such as, but not limited to water, alcohols with fewer than 8 carbon atoms, acetone, acetonitrile, ethylacetate, or miscible mixtures of these solvents to further enrich the active fraction.

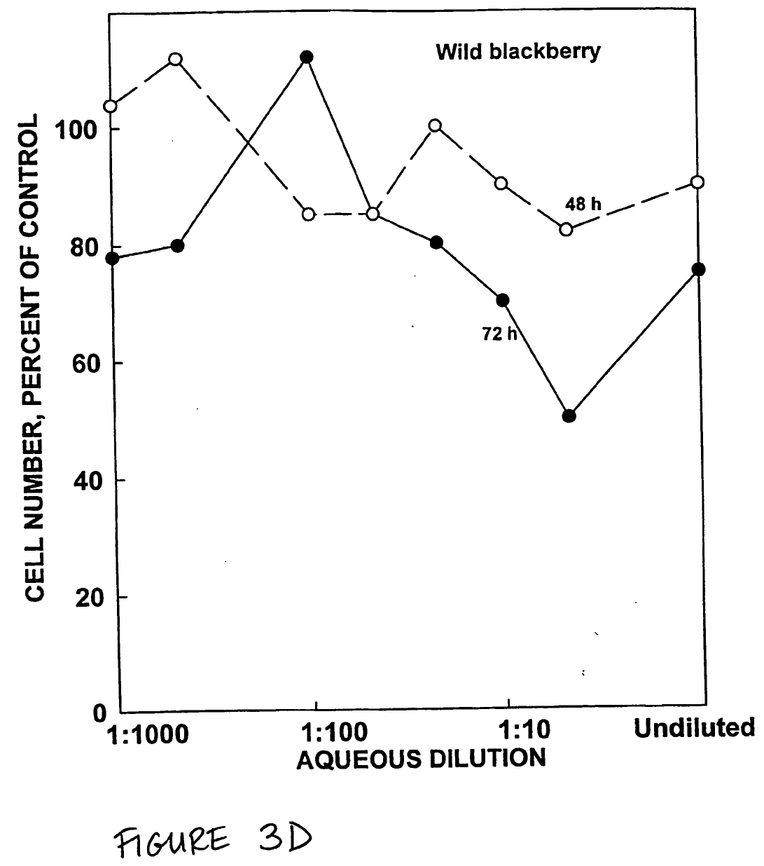

In preferred embodiments, as shown in the results, juice of blue fruits, diluted or concentrated can be used. As shown in the data presented in Table 1 and FIG. 4 in Section 6, pokeberry, greenbriar, and choke cherry have a more potent effect on the inhibition of tNOX activity and the inhibition of HeLa cell growth than the other blue fruits. In a preferred embodiment, the proanthocyanadins are isolated from pokeberry, greenbriar, and/or choke cherry. However, all of the blue fruit extracts presented in Table 1 are effective at inhibiting tNOX activity and HeLa cell growth. The invention is not to be limited to the blue fruits presented herein and the proanthocyanadins of the invention may include those obtained from other natural sources of proanthocyanadins.

### 5.1.3. Combinations of Catechins and Proanthocyanadins

The invention encompasses administration of the catechin formulations listed in Section 5.1.1 and proanthocyanadin formulations listed in Section 5.1.2 in combination. The combination of catechins and proanthocyanadins possess a synergistic effect in the inhibition of tNOX activity and the inhibition of cancer cell growth, as demonstrated in the Example presented in Section 6.

The term “synergistic” as used herein refers to a combination which is more effective than the additive effects of any two or more single agents. A determination of a synergistic interaction between catechins, proanthocyanadins, and optionally, one or more other anti-cancer or therapeutic agents may be based on the results obtained from the NOX assays described in Section 6 infra. The results of these assays are analyzed using Chou and Talalay's combination method and Dose-Effect Analysis with Microcomputers' software in order to obtain a Combination Index (Chou and Talalay, 1984, Adv. Enzyme Regul. 22:27-55 and Chou and Chou, 1987, software and manual, Elsevier Biosoft, Cambridge, UK, pp. 19-64). Combination Index values <1 indicates synergy, values>1 indicate antagonism and values equal to 1 indicate additive effects.

Adjunct administration of the tea catechins and proanthocyanadins of the invention means that the two are administered either as a mixture or sequentially. When administered sequentially, the catechins may be administered before or after the proanthocyanadins, so long as the first administered agent is still providing anti-cancer activity in the animal when the second agent is administered. Any of the modes of administration described infra may be used in combination to deliver the tea catechins and proanthocyanadins.

The present invention is to be understood as embracing all such regimens and the term “adjunct administration” is to be interpreted accordingly. When the tea catechins and proanthocyanadins are administered adjunctively as a mixture, they are preferably given in the form of a composition comprising both agents. Thus, one embodiment of the invention provides for a pharmaceutical composition comprising tea catechins and proanthocyanadins, and optionally, a pharmaceutically acceptable carrier. In another embodiment, when the tea catechins and proanthocyanadins are administered adjunctively as a mixture, they are given in the form of a nutritional composition or dietary supplement comprising tea catechins and proanthocyanadins.

In a specific embodiment, a sustained release catechin formulation, as described infra in Section 5.2. and disclosed in U.S. patent application Ser. No. 09/637,840, which is hereby incorporated by reference in its entirety, is used in combination with a proanthocyanadin formulation, such as but not limited to an extract of one or more blue fruit(s). In a more preferred embodiment, the blue fruit extract is isolated from pokeberry, greenbriar, and/or choke cherry. In another embodiment, the sustained release formulation comprises both catechins and proanthocyanadins.

In another embodiment, a catechin formulation, as described in Section 5.1.1 and disclosed in U.S. patent application Ser. No. 09/537,211, which is hereby incorporated by reference in its entirety, is used in combination with a proanthocyanadin formulation, such as but not limited to an extract of one or more blue fruit(s). In a more preferred embodiment, the blue fruit extract is isolated from pokeberry, greenbriar, or choke cherry. In one embodiment, the catechin formulation is naturally-occurring , i e., endogenous to green tea. In another embodiment, the catechin formulation contains catechin ratios that are not native to green tea.

### 5.1.4. Combinations of Catechins, Proanthocyanadins, and Other Therapeutic Agents

The methods of the invention also encompasses administrating the catechin formulations described in Section 5.1.1 and proanthocyanadin formulations, or pharmaceutically acceptable salts or derivatives thereof, described in Section 5.1.2 in combination with other therapeutic agents, such as anti-cancer drugs, and optionally, pharmaceutically acceptable carriers. The term “pharmaceutically acceptable carrier” refers to a carrier medium that does not interfere with the effectiveness of the biological activity of the active ingredient, is chemically inert and is not toxic to the patient to whom it is administered. The term “pharmaceutically acceptable salts” refers to salts prepared from pharmaceutically acceptable non-toxic acids and bases, including inorganic and organic acids and bases. As used herein the term “pharmaceutically acceptable derivative” refers to any homolog, analog, or fragment corresponding to the formulations as described in Section 5.1 which exhibits anti-cancer activity and is non-toxic to the subject. The term “therapeutic agent” or “anti-cancer agent” refers to any molecule, compound or treatment that assists in the treatment of a cancer or the diseases caused thereby.

The therapeutic agents include, but are not limited to adriamycin and adriamycin conjugates, mechlorethamine, cyclophosphamide, ifosfamide, melphalan, chlorambucil, hexamethylmelamine, thiotepa, busulfan, carmustine, lomustine, semustine, streptozocin, dacarbazine, methotrexate, fluorouacil, floxuridie, cytarabine, mercaptopurine, thioguanine, pentostatin, vinblastine, vincristine, etoposide, teniposide, actinomycin D, daunorubicin, doxorubicin, bleomycin, plicamycin, mitomycin, L-asparaginase, interferon-alpha, cisplatin, carboplatin, mitoxantrone, hydroxyurea, procarbazine, mitotane, aminoglutethimide, prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, acetogenins, e.g., bullatacin, and quassanoids, e.g. simalikalactone D and glaucarubolone, and pharmaceutically acceptable derivatives thereof. The therapeutic agents which have been shown to inhibit tNOX and cancer cell growth include adriamycin, bullatacin, simalikalactone D, and glaucarubolone, descriptions of which are provided in U.S. Pat. No. 5,605,810, which is incorporated by reference in its entirety for all purposes.

Methods of the invention also encompasses administering the catechin formulations and proanthocyanadin formulations, to cancer patients undergoing chemotherapy and/or irradiation for a primary cancer. In a preferred embodiment, use of the catechin formulations, anti-cancer agents, and combinations thereof provides a method for treating the metastasized, i e. secondary cancer, in said patients.

In another embodiment, the catechin and proanthocyanadin formulations of the invention can be administered with a monoclonal antibody directed against tNOX for the treatment or prevention of cancer. An example of such a monoclonal antibody to the human tNOX protein has been described and has been used in the expression cloning of tNOX from HeLa cells (Chueh et al., 1997, Arch. Biochem. Biophys. 342:38-44).

### 5.2. Sustained Release Formulation

The invention further provides tea catechins and/or proanthocyanidins that are formulated as sustained release compositions. As used herein, the term “sustained release formulation” refers to any composition that provides slow, controlled, and/or timed release of one or more active ingredients. In one embodiment, the catechins are formulated as a sustained release formulation and are adjunctively administered with the proanthocyanadins. In another embodiment, the proanthocyanadins are formulated as a sustained release formulation and are adjunctively administered with the catechins. In yet another embodiment, both catechins and proanthocyanadins are formulated as sustained release formulations or as a single sustained release formulation.

In a specific embodiment, the sustained release composition of the invention, when administered to a human, results in circulating levels of the catechins, proanthocyanadins, or both at about 10⁻⁹ and 10⁻⁴ M for at least 48 hours. For the prevention of cancer, the circulating levels of the catechins, proanthocyanadins. or both are preferably maintained at up to 10⁻⁷ M for at least 48 hours in the sera. For the treatment of cancer, the circulating levels of the catechins, proanthocyanadins, or both are preferably maintained at up to 10⁻⁵ M for at least 48 hours in the sera. The levels are either circulating in the patient systemically, or in a preferred embodiment, localized to the tumor, and in a most preferred embodiment, localized to the cell surface of the cancer cells.

It is understood that the catechin and proanthocyanadin levels are maintained over a certain period of time as is desired and can be easily determined by one of skill in the art using this disclosure and available pharmaceutical compendia. In a preferred embodiment, the invention includes a unique feature of administration comprising a sustained release formulation so a constant level of EGCg is maintained between 10⁻⁸ and 10⁻⁶ M between 48 to 96 hours in the sera.

Such sustained and/or timed release formulations may be made by sustained release means or delivery devices that are well known to those of ordinary skill in the art, such as those described in U.S. Pat. Nos. 3,845,770, 3,916,899, 3,536,809, 3,598,123, 4,008,719, 4,710,384, 5,674,533, 5,059,595, 5,591,767, 5,120,548, 5,073,543, 5,639,476, 5,354,556, and 5,733,566, the disclosures of which are each incorporated herein by reference. These compositions can be used to provide slow or sustained release of one or more of the active ingredients using, for example, hydropropylmethyl cellulose, other polymer matrices, gels, permeable membranes, osmotic systems, multilayer coatings, microparticles, liposomes, microspheres, or the like, or a combination thereof to provide the desired release profile in varying proportions. Suitable sustained release formulations known to those of ordinary skill in the art, including those described herein, may be readily selected for use with the compositions of the invention. Thus, single unit dosage forms suitable for oral administration, such as, but not limited to, tablets, capsules, gelcaps, caplets, powders, and the like, that are adapted for sustained release are encompassed by the present invention.

In a highly preferred embodiment, the sustained release formulation contains active ingredients such as, but not limited to, microcrystalline cellulose, maltodextrine, ethylcellulose, and magnesium stearate. In yet another highly preferred embodiment, the formulation is synthesized with a CapsuDar® SR (Biodar, Yavne, Israel) microencapsulation which consists of the active ingredients microcrystalline cellulose, maltodextrine, ethylcellulose, and magnesium stearate.

As described above, all known methods for encapsulation which are compatible with the properties of tea catechins and proanthocyanadins are compassed by this invention. The sustained release formulation is encapsulated by coating particles or granules of the composition of the invention with varying thicknesses of slowly soluble polymers or by microencapsulation. In a preferred embodiment, the sustained release formulation is encapsulated with a coating material of varying thickness (e.g., about 1 micron to 200 microns) that allows the dissolution of the pharmaceutical composition about 48 hours to about 72 hours after administration to a mammal. In another embodiment, the coating material is a food approved additive. In yet another embodiment, the coating material is sold under the trademark Eudragit RS or RL (Rohm Pharma, Germany).

In another embodiment, the sustained release formulation is a matrix dissolution device, which is prepared by compressing the drug with a slowly soluble polymer carrier into a tablet. In one preferred embodiment, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, have a relatively high percentage of plasticizer in the coating in order to permit sufficient flexibility to prevent substantial breakage during compression. The specific amount of plasticizer varies depending on the nature of the coating and the particular plasticizer used. The amount may be readily determined empirically by testing the release characteristics of the tablets formed. If the medicament is being released too quickly, then more plasticizer is used. Release characteristics are also a finction of the thickness of the coating. When substantial amounts of plasticizer are used, the sustained released capacity of the coating diminishes. Thus, the thickness of the coating may be increased slightly to make up for an increase in the amount of plasticizer. Generally, the plasticizer in such an embodiment will be present in an amount of about 15 to 30 percent of the sustained release material in the coating, preferably 20 to 25 percent and the amount of coating will be from 10 to 25 percent of the weight of active material, preferably 15 to 20 percent. Any conventional pharmaceutically acceptable plasticizer may be incorporated into the coating.

The disclosure of U.S. patent application Ser. No. 09/637,840, which is directed to the use of sustained release formulations of catechins for the treatment and prevention of cancer, is hereby incorporated by reference in its entirety.

### 5.3. Target Cancers

Cancers that can be prevented or treated by the methods of the present invention include, but not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

In a preferred embodiment, the cancer is one where circulating levels of tNOX are present in the sera of patients suffering from said cancer, e.g., rectal carcinoma, colon carcinoma, breast carcinoma, ovarian carcinoma, small cell lung carcinoma, colon carcinoma, chronic lymphocytic carcinoma, hairy cell leukemia, osophogeal carcinoma, prostate carcinoma, breast cancer, myeloma, and lymphoma, see e.g., U.S. Pat. No. 5,605,810, which is incorporated by reference in its entirety.

In a preferred embodiment, the patient already has cancer and is undergoing treatment for said cancer. In a specific embodiment, the patient already has cancer but no metastasis. i.e., secondary cancer. In another specific embodiment, the patient already has cancer plus a metastatic cancer. In another specific embodiment, the patient having a cancer is immunosuppressed by reason of having undergone anti-cancer therapy (e.g., chemotherapy or radiation) prior to administration of the catechin complexes of the invention. In yet another embodiment, the patient is a post-treatment or post-operative cancer patient.

In another specific embodiment, the cancer is a tumor. In a preferred embodiment, the tumor is a tumor of epithelial tissue, lymphoid tissue, connective tissue, bone, or central nervous system.

### 5.4. Modes of Administration

### 5.4.1. Sustained Release Formulation

The catechins and proanthocyanadins of the invention can be formulated as a sustained and/or timed release formulation as described in Section 5.2. The levels of circulating catechin and proanthocyanadin compositions must be maintained above some minimum therapeutic dose to reduce the number of cancer cells or to prevent cancer. The reduction in the number of cancer cells can be the result of cell death or apoptosis. The reduction in the number of cancer cells can be a result of inhibition of cell growth, or cell growth arrest.

All sustained release products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: (1) extended activity of the composition; (2) reduced dosage frequency; and (3) increased patient compliance. In addition, sustained release formulations can be used to affect the time of onset of action or other characteristics, such as blood levels of the composition, and thus can affect the occurrence of side effects.

The sustained release formulations of the invention are designed to initially release an amount of the therapeutic composition that promptly produces the desired therapeutic effect, and gradually and continually release other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds. The term “sustained release component” in the context of the present invention is defined herein as a compound or compounds, including, but not limited to, polymers, polymer matrices, gels, permeable membranes, liposomes, microspheres, or the like, or a combination thereof, that facilitates the sustained release of the active ingredient.

### 5.6.2. Modes of Administration of Water-Soluble Compositions

If the composition is water-soluble, then it may be formulated in an appropriate buffer, for example, phosphate buffered saline or other physiologically compatible solutions. Alternatively, if the resulting composition has poor solubility in aqueous solvents, then it may be formulated with a non-ionic surfactant such as Tween, or polyethylene glycol. Thus, the compounds and their physiologically acceptable solvates may be formulated for administration by inhalation or insufflation (either through the mouth or the nose) or oral, buccal, parenteral, rectal administration or, in the case of tumors, directly injected into a solid tumor.

### 5.6.3. Oral Administration

For oral administration, the composition may be in liquid form, (e.g., solutions, syrups or suspensions), or may be presented as a drug product (e.g., capsule or powder) for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. In a preferred embodiment, the composition may take the form of a capsule or powder to be dissolved in a liquid for oral consumption.

Preparations for oral administration may be suitably formulated to give controlled release of the active compound. In a preferred embodiment, the compounds of the present invention are formulated as controlled release powders of discrete micro-particles which can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556 which is incorporated by reference in its entirety. Particularly suitable polymers include those described, but not limited to, those described in column 3, line 46-column 4, line 8 of U.S. Pat. No. 5,354,556 which is incorporated by reference in its entirety.

### 5.6.4. Buccal Administration

For buccal administration, the compositions may take the form of tablets or lozenges formulated in conventional manner.

### 5.6.5. Parenteral Administration

The compositions of the invention may be formulated for parenteral administration, e.g., by intramuscular injections or implants for subcutaneous tissues and various body cavities and transdermal devices.

Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In a preferred embodiment, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil solutions and suspensions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachnis, maize, almond, cottonseed, and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

### 5.6.6. Rectal Administration

The compositions may also be formulated in rectal compositions such as suppositories or retention enemas, e.g., containing conventional suppository bases such as cocoa butter or other glycerides.

### 5.6.7. Packs and Kits

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers having therapeutically or prophylactically effective amounts of the catechin and proanthocyanadin compositions in pharmaceutically acceptable form. The catechin and proanthocyanadin composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the complex, and/or a packaged alcohol pad. Instructions are optionally included for administration of catechin/proanthocyanadin composition by a clinician or by the patient.

### 5.7. Dosage

### 5.7.1. Catechins and Proanthocyanadins as a Dietary or Nutritional Supplement

In one embodiment of this invention, a formulation comprising catechins and proanthocyanadins may be used as a dietary or nutritional supplement. In this embodiment, the total daily dose ranges of the active catechins and proanthocyanadins for the conditions described herein are generally from about 10 mg to about 800 mg of catechins and from about 5 to 400 mg of proanthocyanadins administered in divided doses administered parenterally or orally. A preferred total daily dose is from about 50 mg to about 400 mg of the active catechins and from about 25 to 200 mg of proanthocyanadins. The dosages of proanthocyanadins described herein are also understand by one of skill in the art to be an equivalent amount of blue fruit extract.

In another embodiment, a total daily dose of a formulation may be used as a dietary supplement is about 10 mg to about 800 mg of active catechins and from about 5 to 400 mg of proanthocyanadins administered twice daily (e.g., in the morning and the evening) at a dose of about 5 mg to about 400 mg and from about 2 to 200 mg of proanthocyanadins. The dosage forms and compositions may comprise any of the forms and compositions described supra. In a preferred embodiment, the formulation comprising catechins and proanthocyanadins is a tablet, capsule, gel, or a liquid-soluble powder.

### 5.7.2. Catechins and Proanthocyanadins as a Therapeutic

In another embodiment of the invention, the magnitude of a therapeutic dose of catechins and proanthocyanadins in the acute or chronic management of cancer will vary with the severity of the condition to be treated and the route of administration. The dose, and dose frequency, will also vary according to the age, body weight, condition and response of the individual patient, and the particular catechin and proanthocyanadin combination used. All combinations described in the specification are encompassed as therapeutic, active catechin and proanthocyanadin mixtures and it is understood that one of skill in the art would be able to determine a proper dosage of particular catechin and proanthocyanadin mixtures using the parameters provided in the invention. Furthermore, one of ordinary skill in the art would be able to vary the dose of the proanthocyanadins relative to the amounts of catechins present, based on the guidance provided throughout the invention, particularly as described in Example 6.

In general, the total daily dose ranges of the active catechins for the conditions described herein are generally from about 10 mg to about 1000 mg and from about 5 to 500 mg of proanthocyanadins administered in divided doses administered parenterally or orally or topically. A preferred total daily dose is from about 200 mg to about 600 mg of the active catechins and from about 100 to 300 mg of proanthocyanadins. The dosages of proanthocyanadins described herein are also understand by one of skill in the art to be an equivalent amount of blue fruit extract.

For example, in one embodiment, the daily dose ranges of catechins for the conditions described herein are generally from about 10 to about 100 mg per kg weight and from about 5 to 50 mg of proanthocyanadins. Preferably the catechin and proanthocyanadin formulation of the invention is given daily until remission, followed by two to ten additional cycles, each lasting about 60 days in duration. When the dose is administered orally, a sustained release formulation is preferred so that a fairly constant level of catechins is provided over the course of treatment, which is generally at least 48 hours and preferably at least 96 hours per cycle. As the catechins and proanthocyanadins are not particularly toxic, the formulation may be administered for as long as necessary to achieve the desired therapeutic effect.

In the case where an intravenous injection or infusion composition is employed, a suitable dosage range for use is, e.g., from 1 to about 10 mg per kg body weight of catechins and from about 0.5 to 5 mg per kg body weight of proanthocyanadins total daily.

For treatment of solid tumors, a preferred dosing regimen involves intravenous infusion of about 1 to about 10 mg per kg body weight of catechins and from about 0.5 to 5 mg per kg body weight of proanthocyanadins extract. This daily treatment protocol is repeated once per month until the tumor growth tumor is inhibited or when the tumor shows signs of regression.

In an alternative embodiment of the invention, the effect of the therapy with catechins and proanthocyanadins on cancer treatment can be monitored by any methods known in the art, including but not limited to monitoring circulating tNOX activity in patient sera, as well as more traditional approaches such as determining levels of tumor specific antigens and putative biomarkers, e.g., carcinoembryonic antigens (CEA), alpha-fetoprotein; and changes in morphology and/or size using computed tomographic scan and/or sonogram.

Desirable blood levels may be maintained by a continuous infusion of catechins and proanthocyanadins as ascertained by plasma levels. It should be noted that the attending physician would also know how to and when to adjust treatment to higher levels if the clinical response is not adequate (precluding toxic side effects, if any).

Again, any suitable route of administration may be employed for providing the patient with an effective dosage of the catechin and proanthocyanadin combination of this invention. Dosage forms include tablets, troches, cachet, dispersions, suspensions, solutions, capsules, gel caps, caplets, compressed tablets, sustained release devices, patches, and the like.

The pharmaceutical compositions of the present invention comprise catechins and proanthocyanadins as the active ingredients, as well as pharmaceutically acceptable salts thereof, and may also contain a pharmaceutically acceptable carrier, and optionally, other therapeutic ingredients. The term “pharmaceutically acceptable salts” refers to salts prepared from pharmaceutically acceptable non-toxic acids and bases, including inorganic and organic acids and bases.

The pharmaceutical compositions include compositions suitable for oral and parenteral (including subcutaneous, intramuscular, intrathecal, intravenous, and other injectables) routes, although the most suitable route in any given case will depend on the nature and severity of the condition being treated.

In addition, the catechin and proanthocyanadin carrier could be delivered via charged and uncharged matrices used as drug delivery devices such as cellulose acetate membranes, also through targeted delivery systems such as fusogenic liposomes attached to antibodies or specific antigens.

In practical use, catechins and proanthocyanadins can be combined as the active ingredient(s) in intimate admixture with a pharmaceutical carrier according to conventional pharmaceutical compounding techniques. The carrier may take a wide variety of forms depending on the form of preparation desired for administration, e.g., oral or parenteral (including tablets, capsules, powders, intravenous injections or infusions). In preparing the compositions for oral dosage form any of the usual pharmaceutical media may be employed, e.g., water, glycols, oils, alcohols, flavoring agents, preservatives, coloring agents, and the like; in the case of oral liquid preparations, e.g., suspensions, solutions, elixirs, liposomes and aerosols; starches, sugars, micro-crystalline cellulose, diluents, granulating agents, lubricants, binders, disintegrating agents, and the like in the case of oral solid preparations e.g., powders, capsules, and tablets. In preparing the compositions for parenteral dosage form, such as intravenous injection or infusion, similar pharmaceutical media may be employed, e.g., water, glycols, oils, buffers, sugar, preservatives and the like know to those skilled in the art. Examples of such parenteral compositions include, but are not limited to Dextrose 5% (w/v), normal saline or other solutions. The total dose of the catechins and proanthocyanadins may be administered in a vial of intravenous fluid, e.g., ranging from about 0.01 to about 100 mg per kg body weight of catechins and from about 0.005 to 50 mg per kg body weight of proanthocyanadins. The volume of dilution fluid will vary according to the total dose administered and over the length of the period of time of administration.

An exemplary course of treatment of a patient with cancer or solid cancer can involve daily administration by intravenous infusion of catechins and proanthocyanadins in an aqueous solution at a daily dose of about 1 to about 10 mg of the catechins and from about 0.5 to 5 mg of proanthocyanadins per kg of body weight of the patient. The course of treatment may be repeated for up to ten times over approximately 10 months with a break of about three to six weeks in between courses. The post-remission course of treatment involves infusion of catechins at a daily dose of about 0.01 to about 1 mg and from about 0.005 to 0.5 mg of proanthocyanadins per kg of body weight of the patient on a daily or weekdays-only basis for a cumulative total of 25 days.

In another embodiment, the invention encompasses the daily dose ranges of catechins for the conditions described herein are generally from about 0.1 to about 15 mg and from about 0.05 to 8 mg of proanthocyanadins per kg body weight administered in divided doses administered orally. Preferably the catechin and proanthocyanadin formulation of the invention is given daily, or until remission, followed by two to ten additional cycles, each lasting about 60 days in duration. When the dose is administered orally, a sustained release formulation is preferred so that a fairly constant level of catechins and proanthocyanadins is provided over the course of treatment, which is generally at least 48 hours and preferably at least 96 hours per cycle. As the catechins and proanthocyanadins are not particularly toxic, the formulation may be administered for as long as necessary to achieve the desired therapeutic effect. In the case where an intravenous injection or infusion composition is employed, a suitable dosage range for use is, e.g., from about 0.01 to about 1.5 mg per kg body weight of catechins and from about 0.005 to 1 mg of proanthocyanadins total daily.

For treatment of solid tumors, a preferred dosing regimen involves intravenous infusion of the active catechins of the invention, as described above, in the amount of about 0.01 to about 10 mg and from about 0.005 to 5 mg of proanthocyanadins per kg body weight per day. This daily treatment protocol is repeated once per month until the tumor growth tumor is inhibited or when the tumor shows signs of regression.

The effect of the therapy with catechins and proanthocyanadins on cancer treatment can be monitored by methods stated supra. Similarly, pharmaceutical compositions and routes of administration are similar as those described supra.

For the purposes described above, the invention also encompasses methods for monitoring patient response to tea catechins and proanthocyanadins. By monitoring circulating tNOX activity in patient sera, it will be possible to determine therapeutic dosages and to monitor therapeutic benefit from tea catechins and proanthocyanadins. The response of neoplastic cells to the subject compositions may be monitored by assaying the blood or urine of the patient for the NOX activity that is responsive to the catechin and proanthocyanadin compositions, i.e., tNOX. Various assays may be used to monitor activity, such as a NOX assay for neoplasia determination see e.g., U.S. Pat. No. 5,605,810. By following the above monitoring procedures, an effective dosage of the subject compositions may be administered in accordance with the requirement of an individual patient.

### 6. EXAMPLE

### Synergistic Effect of Catechins and Proanthocyanadins

### 6.1. Materials and Methods

HeLa (ATCC CCL2) cells were grown in 150 cm² flasks in Minimal Essential Medium (Gibco), pH 7.4, at 37° C. with 10% bovine calf serum (heat inactivated, plus 50 mg/l gentamicin sulfate (Sigma). A mouse mammary tumor subpopulation line 4T1 derived from a BALB/cfC3H mouse (Miller et al., 1981, Brit. J. Cancer 56: 561 and Miller et al., 1990, Invasion Metastasis 10: 101) was grown in DME 10, Dulbecco's modified Eagle's medium supplemented with 5% fetal calf serum, 5% newborn calf serum, 1 mM mixed non-essential amino acids, 2 mM L-glutamine, penicillin (100 units/ml), and streptomycin (100 μg/ml).

Cells were quantitated using a standard cytotoxicity assay (Matthews, N. and Neale, M. L. In Lymphokines and Interferons. A Practical Approach. Clemens, M. J. et al., eds. IRL Press, Oxford, p. 221 (1987)).

NADH oxidase activity was monitored and measured by virtue of the decrease in absorbance at 340 nm wavelength. A millimolar extinction coefficient of 6.22 for NADH was used to calculate rates of NADH oxidation. Additional tests that may be used include those such as those described by Morré, 1994, Bioenerg. Biomemb. 26:421, and Chueh et al., 1997, J. Biol. Chem. 272:11221, which are incorporated by reference in their entireties.

The blue fruits are crushed (e.g., with a garlic press) to obtain the blue fruit extracts, and the subsequent dilutions of the extracts are indicated as ratios (e.g., 1:500 indicating a 500 part dilution of the extract in water or a buffer) in the figures and table.

### 6.2. Results

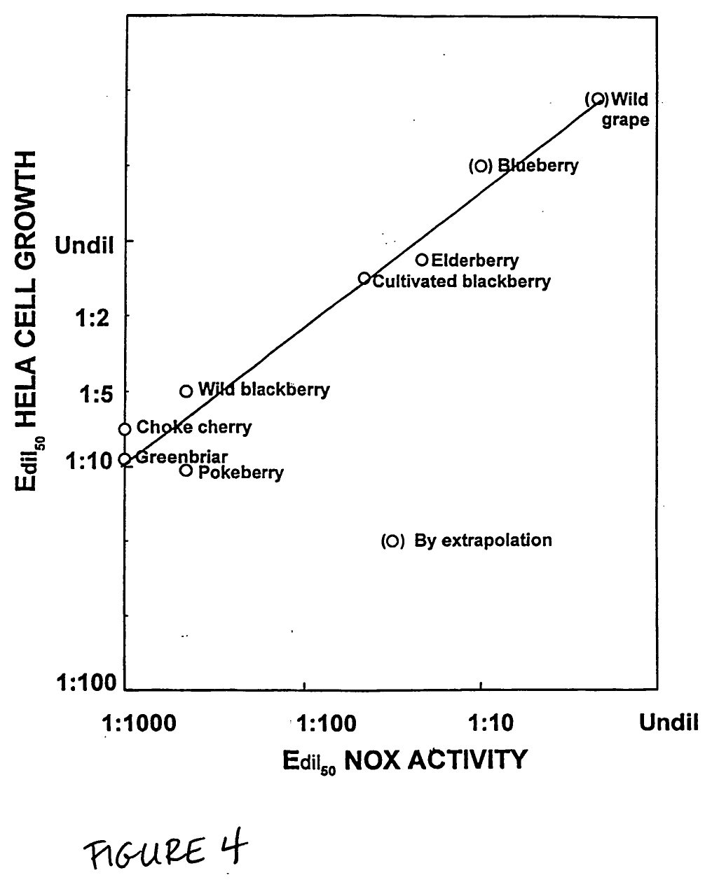

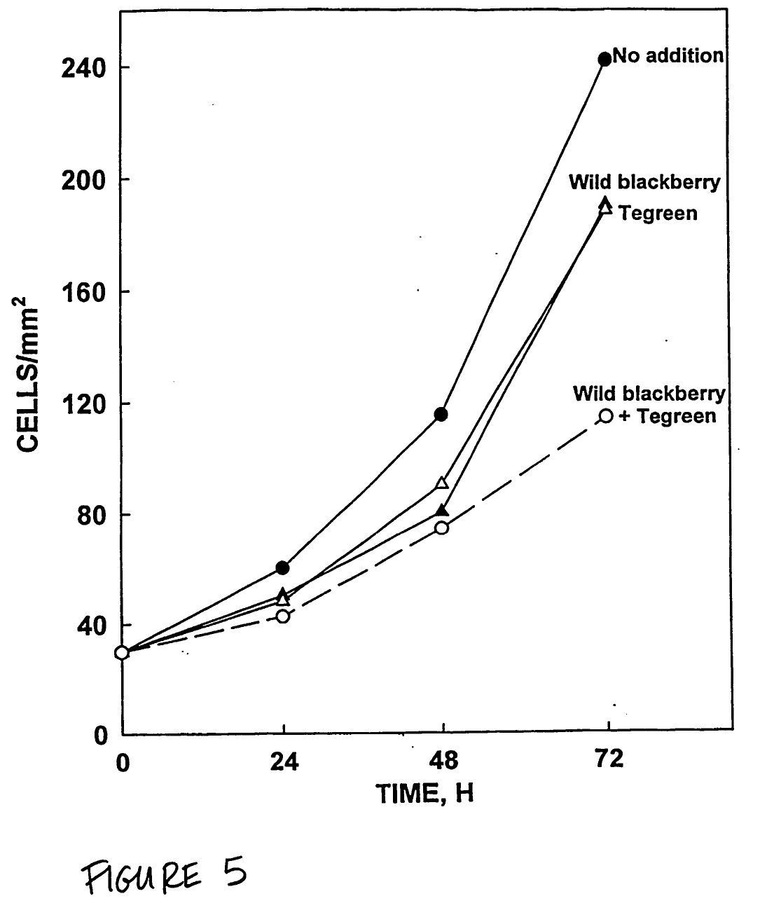

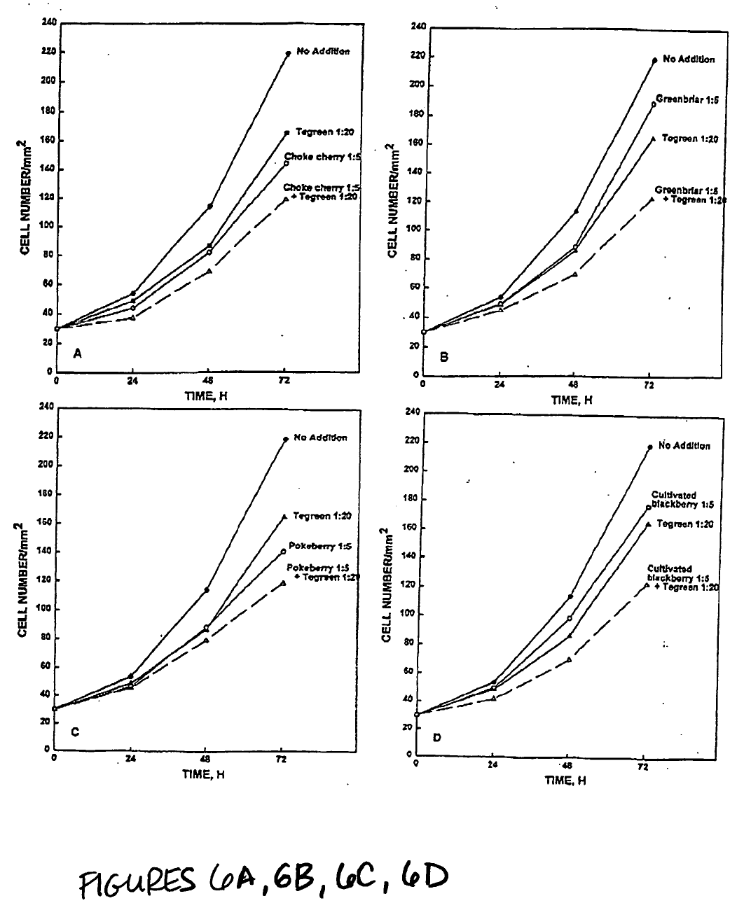

Data presented herein illustrate that proanthocyanadins i.e., the blue fruit extracts, inhibit both NADH oxidase activity, i.e., tNOX, and growth of human cervical carcinoma cells, i.e. HeLa cells. As shown in FIGS. 5 and 6, a synergistic effect exists between tea catechins and the proanthocyanadins of the blue fruit extracts.

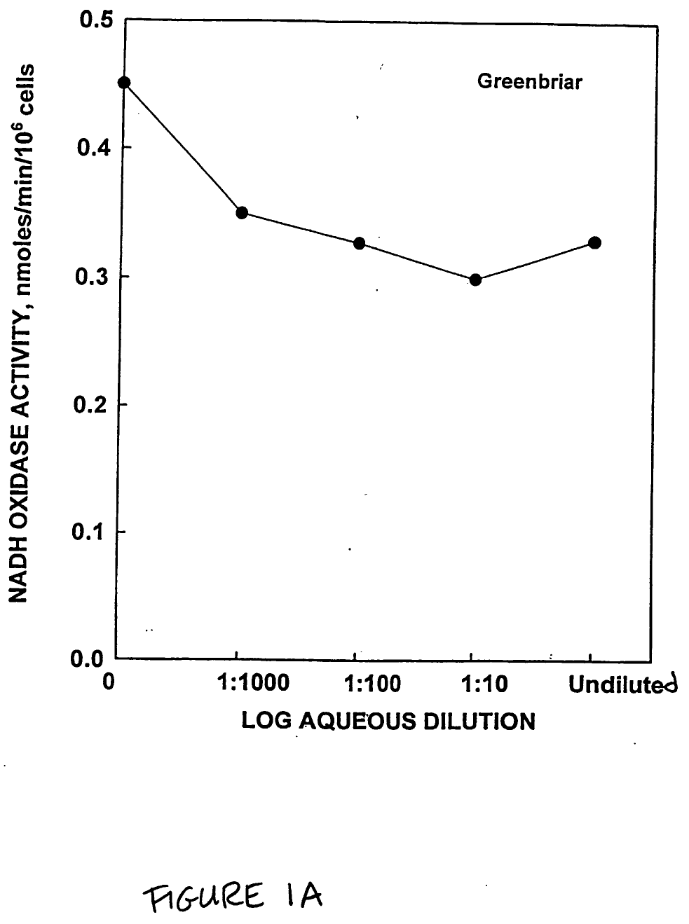

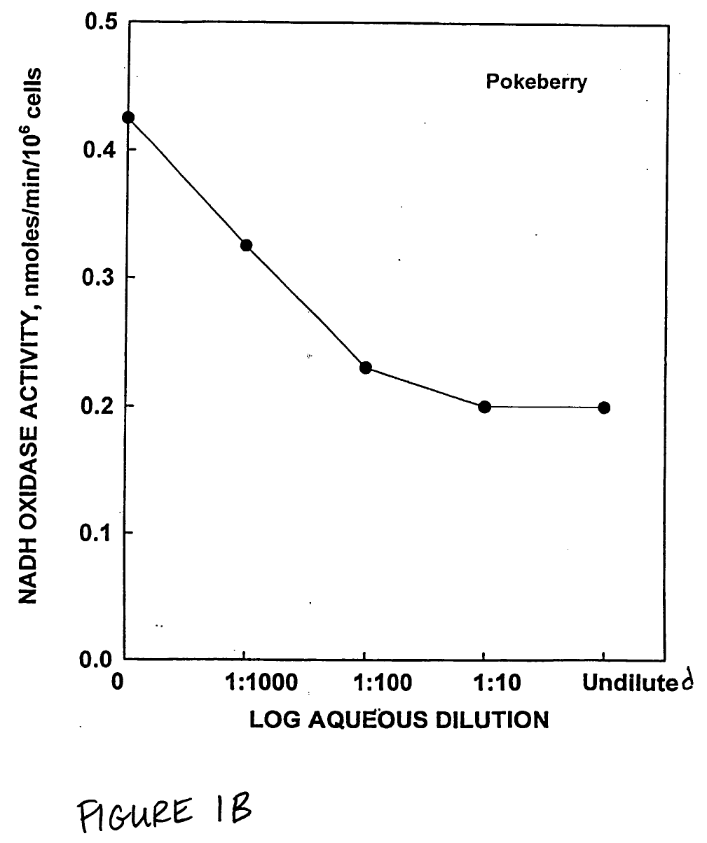

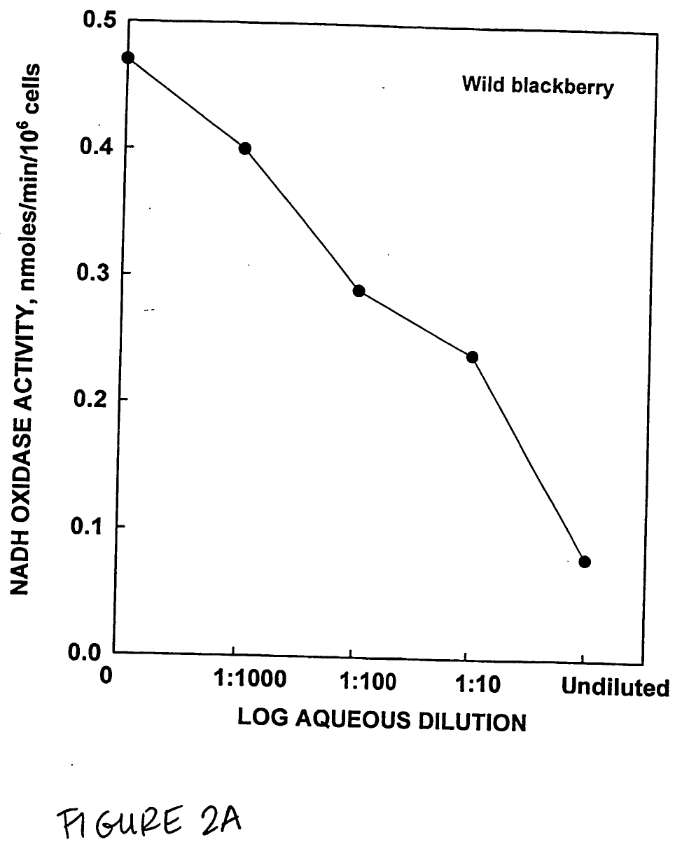

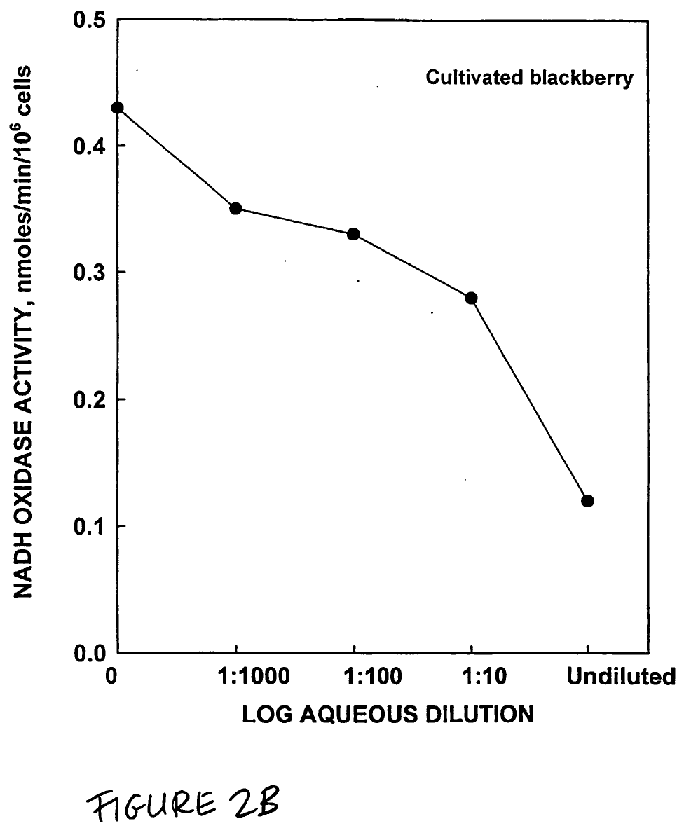

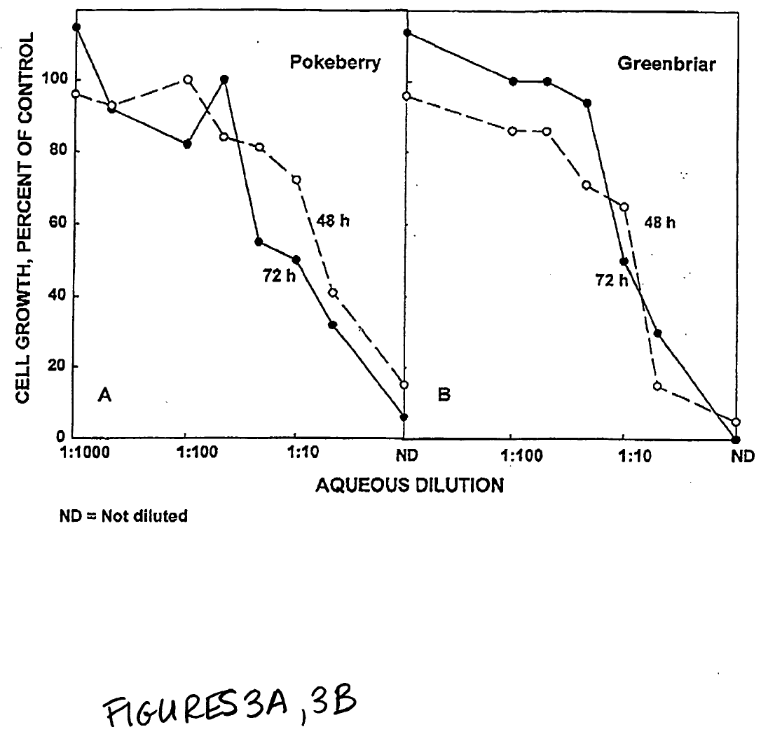

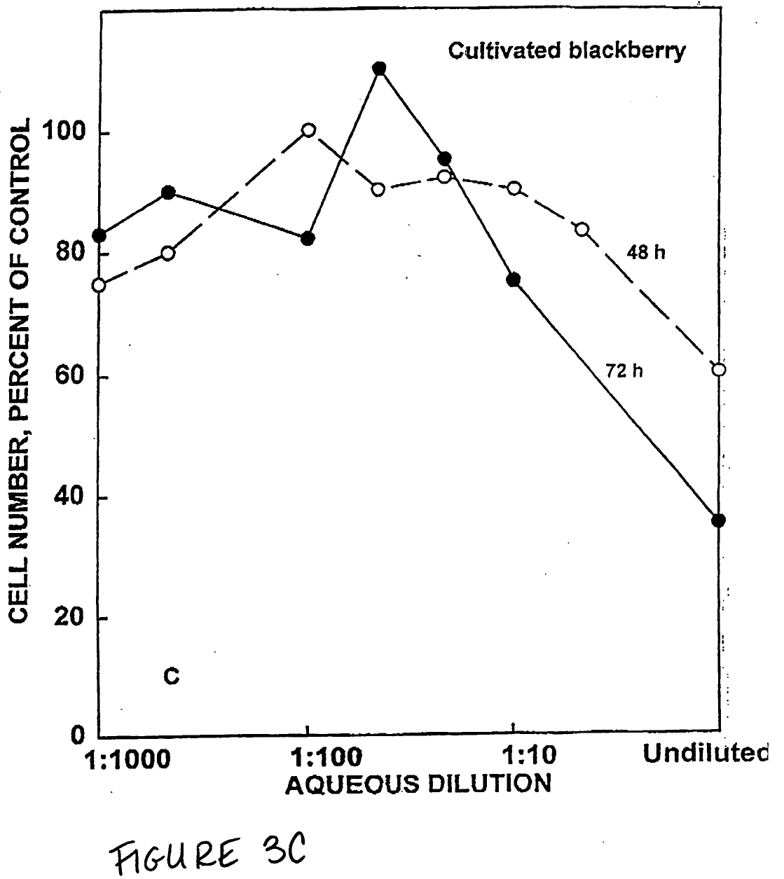

As shown in FIGS. 1, 2, and 3, the juice of greenbriar (Smilax glauca), pokeberry (Phytolacca americana), and wild and cultivated blackberry (Rubus spp) inhibit both tNOX activity and the growth of cultured HeLa cells. The data in FIG. 4 and Table 1 indicate that pokeberry, greenbriar, and choke cherry are more effective than the other blue fruits tested for inhibiting tNOX activity and HeLa cell growth among the blue fruits tested. Despite the varying specific activities of the blue fruit extracts that are tested, all of the blue fruit extracts that are tested are effective for inhibiting tNOX activity and HeLa cell growth.

FIGS. 5 and 6 illustrate the effect on HeLa cell growth by a combination of a commercially supplied tea concentrate (Tegreen™, Pharmanex, Brisbane, Calif.) and the extract from wild and cultured blackberry, choke cherry, greenbriar, and pokeberry. A synergistic effect between green tea catechins and blue fruit proanthocyanadins became evident after 72 hours, as demonstrated in the effect of the mixture of Tegreen™ and choke cherry, greenbriar, and pokeberry. A more pronounced synergistic effect is present in the mixture of Tegreen™ and cultivated blackberry extract and even greater synergistic effect is evidenced in the mixture of Tegreen™ and wild blackberry extract on HeLa cell growth, as shown in FIG. 5.

TABLE 1
Edil₅₀ (aqueous dilution to give 50% inhibition) for
inhibition of tNOX activity and growth of cultured HeLa
(human cervical carcinoma cells) in culture (96 well plate assay)
for juices from eight blue fruits.

Edil₅₀

| | Inhibition of | Inhibition of |
|---|---|---|
| | t-NOX activity | HeLa Growth (72 h) |
| Wild blackberry | 1:500 | 1:5 |
| Cultivated blackberry | 1:50 | 1:1.5 |
| Elderberry | 1:20 | Undiluted |
| Blueberry | 1:10 | Undiluted (20%) |
| Choke cherry | 1:1000 | 1:6 |
| Pokeberry | 1:5000 | 1:10 |
| Wild grape | 1:2 | Undetermined |
| Greenbriar | 1:1000 | 1:10 |

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

What is claimed is:

1. A method for prevention or treatment of a cancer in a mammal, said method comprising administering to a mammal a composition comprising tea catechins and proanthocyanadins, in amounts effective to prevent or treat the cancer.

2. A method for prevention or treatment of a cancer in a mammal, said method comprising administering to a mammal a composition comprising tea catechins adjunctively with a composition comprising proanthocyanadins, wherein the amounts of tea catechins and proanthocyanidins administered are effective to prevent or treat the cancer.

3. The method of claim 1 or 2 wherein the mammal is a human.

4. The method of claim 1 or 2 wherein the cancer is selected from a group comprising rectal carcinoma, colon carcinoma, breast carcinoma, ovarian carcinoma, small cell lung carcinoma, colon carcinoma, chronic lymphocytic carcinoma, hairy cell leukemia, osophogeal carcinoma, prostate carcinoma, breast cancer, myeloma, and lymphoma.

5. The method of claim 1 or 2 wherein the cancer is a tumor and wherein said tumor is a tumor of epithelial tissue, lymphoid tissue, connective tissue, bone, or central nervous system.

6. The method of claim 3 wherein the human is immunosuppressed by reason of having undergone anti-cancer therapy.

7. The method of claim 1 or 2 wherein the cancer is a metastases.

8. The method of claim 1 or 2 wherein the level of tea catechins and proanthocyanadins is maintained at constant levels in the sera for at least 48 hours.

9. The method of claim 1 wherein said composition of tea catechins and proanthocyanadins is a sustained release formulation and comprises at least one component which controls the release of said catechins and/or proanthocyanadins.

10. The method of claim 2 wherein said composition of tea catechins or said composition of proanthocyanadins or both are sustained release formulations and comprises at least one component which controls the release of said catechins and/or proanthocyanadins.

11. A method for treatment of a cancer in a mammal, said method comprising administering to a mammal a composition comprising tea catechins and proanthocyanadins, or pharmaceutically acceptable salts thereof, in combination with an effective amount of at least one other anti-cancer agent, in amounts effective to treat the cancer, wherein the cancer is a type having cancer cells which express tNOX.

12. The method of claim 11 in which said other anti-cancer agent is selected from a group consisting of adriamycin and adriamycin conjugates, mechlorethamine, cyclophosphamide, ifosfamide, melphalan, chlorambucil, hexamethylmelamine, thiotepa, busulfan, carmustine, lomustine, semustine, streptozocin, dacarbazine, methotrexate, fluorouacil, floxuridie, cytarabine, mercaptopurine, thioguanine, pentostatin, vinblastine, vincristine, etoposide, teniposide, actinomycin D, daunorubicin, doxorubicin, bleomycin, plicamycin, mitomycin, L-asparaginase, interferon-alpha, cisplatin, carboplatin, mitoxantrone, hydroxyurea, procarbazine, mitotane, aminoglutethimide, prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, acetogenins, e.g., bullatacin, and quassanoids, e.g. simalikalactone D and glaucarubolone, and pharmaceutically acceptable derivatives thereof.

13. The method of claim 1, 2, 7, or 11 in which said administration is made via an implantation device.

14. The method of claim 1, 2, 7, or 11 in which said administration is made with a sustained release formulation.

15. The method of claim 5 in which said administration is made parenterally, orally, or directly into the tumor.

16. The method of claim 1, 2, 7 or 11 wherein said proanthocyanadins are extracted from a blue fruit.

17. The method of claim 16 wherein said blue fruit is selected from the group consisting of cultured blackberries (Rubus spp.) (Rosaceae), wild blackberries (Rubus spp.) (Rosaceae), blue berries (Vaccinium sp.) (Ericaceae), elderberry (Sambucus canadensis) (Caprifoliaceae), choke cherry (Prunus virginiana) (Rosaceae), pokeberry (Phytolacca americana) (Phytolaccaceae), wild grape (fox grape) (Vitis labrusca) (Vitaceae), and green briar (Smilax glauca) (Liliaceae).

18. A dietary or nutritional supplement comprising an effective amount of tea catechins and proanthocyanadins to inhibit and/or treat cancer in a mammal.

19. The supplement of claim 18 wherein the supplement is a sustained release formulation comprising tea catechins and/or proanthocyanadins and at least one component which controls release of said catechins and/or proanthocyanadins.

20. The supplement of claim 18 wherein the supplement is formulated as an oral preparation comprising tablets or powders.

21. The supplement of claim 18 wherein the supplement is formulated as a sterile preparation.

22. The supplement of claim 18 wherein the supplement is formulated as a parenteral solution.

23. The supplement of claim 18 wherein said proanthocyanadins are extracted from a blue fruit.

24. The supplement of claim 23 wherein said blue fruit is selected from the group consisting of cultured blackberries (Rubus spp.) (Rosaceae), wild blackberries (Rubus spp.) (Rosaceae), blue berries (Vaccinium sp.) (Ericaceae), elderberry (Sambucus canadensis) (Caprifoliaceae), choke cherry (Prunus virginiana) (Rosaceae), pokeberry (Phytolacca americana) (Phytolaccaceae), wild grape (fox grape) (Vitis labrusca) (Vitaceae), and green briar (Smilax glauca) (Liliaceae).

25. The supplement of claim 18 wherein daily dosage of the catechins is about 10 mg to about 800 mg and daily dosage of the proanthocyanadins is about 5 mg to about 400 mg.

26. The supplement of claim 25 wherein daily dosage of the catechins is about 50 mg to about 400 mg and daily dosage of the proanthocyanadins is about 25 mg to about 200 mg.

27. A pharmaceutical composition comprising an effective amount of tea catechins and proanthocyanadins to inhibit and/or treat cancer in a mammal.

28. The composition of claim 27 wherein the composition is a sustained release formulation comprising tea catechins and/or proanthocyanadins and at least one component which controls release of said catechins and/or proanthocyanadins.

29. The composition of claim 27 wherein the composition is formulated as an oral preparation comprising tablets or powders.

30. The composition of claim 27 wherein the composition is formulated as a sterile preparation.

31. The composition of claim 27 wherein the composition is formulated as a parenteral solution.

32. The composition of claim 27 wherein said proanthocyanadins are extracted from a blue fruit.

33. The composition of claim 32 wherein said blue fruit is selected from the group consisting of cultured blackberries (Rubus spp.) (Rosaceae), wild blackberries (Rubus spp.) (Rosaceae), blue berries (Vaccinium sp.) (Ericaceae), elderberry (Sambucus canadensis) (Caprifoliaceae), choke cherry (Prunus virginiana) (Rosaceae), pokeberry (Phytolacca americana) (Phytolaccaceae), wild grape (fox grape) (Vitis labrusca) (Vitaceae), and green briar (Smilax glauca) (Liliaceae).

34. The composition of claim 27 wherein daily dosage of the catechins is about 10 mg to about 1000 mg and daily dosage of the proanthocyanadins is about 5 mg to about 500 mg.

35. The composition of claim 34 wherein daily dosage of the catechins is about 200 mg to about 600 mg and daily dosage of the proanthocyanadins is about 100 mg to about 300 mg.
